(19) United States
(12) Patent Application Publication
(10) Pub. No.: US 2026/0069860 A1
(43) Pub. Date: Mar. 12, 2026

(54) SYSTEMS AND METHODS FOR PERIPHERAL NERVE STIMULATION USING MICROBURSTS OR NERVE MAPPING PARADIGMS

(71) Applicant: Boston Scientific Neuromodulation Corporation, Valencia, CA (US)

(72) Inventors: Hari Hara Subramanian, Valencia, CA (US); Chirag Shah, Valencia, CA (US); Adarsh Jayakumar, Valencia, CA (US); Nathan Maas, Simi Valley, CA (US); Sarvani Grandhe, Valencia, CA (US)

(21) Appl. No.: 19/317,794

(22) Filed: Sep. 3, 2025

Related U.S. Application Data
(60) Provisional application No. 63/691,918, filed on Sep. 6, 2024.

(51) Int. Cl. A61N 1/05; A61N 1/36; A61N 1/372

(52) U.S. Cl. CPC A61N 1/0556; A61N 1/36125; A61N 1/36157; A61N 1/36175; A61N 1/36178; A61N 1/36185; A61N 1/37247

(57) ABSTRACT

A system for stimulating a nerve includes a control module and a cuff lead with electrodes. The control module includes stimulation circuitry, a memory, and a processor configured to direct the stimulation circuitry to deliver a macroburst to the cuff lead for stimulation using the electrodes. The macroburst includes multiple microbursts. Each microburst includes pulses arranged in a temporal sequence. Each pulse has a pulse width of no more than 100 μs and a total amplitude of no more than 100 μA. A system for mapping a nerve can include a processor for directing the control module to deliver stimulation pulses according to at least one nerve mapping paradigm that includes a temporal sequence of pulses; associating stimulation effects with the electrodes of the cuff lead based on the temporal sequence; and mapping the electrodes to one or more axons, fascicles, nerve fibers, tracts of the nerve, or peripheral organs.

### CROSS-REFERENCE TO RELATED APPLICATIONS

This application claims the benefit under 35 U.S.C. §119(e) of U.S. Provisional Patent Application Ser. No. 63/691,918, filed Sep. 6, 2024, which is incorporated herein by reference.

### FIELD

The present disclosure is directed to the area of peripheral nerve stimulation systems and methods of making and using the systems. The present disclosure is also directed to programming and stimulation using macrobursts, having multiple microbursts, or nerve mapping paradigms.

### BACKGROUND

Implantable electrical stimulation systems have proven therapeutic in a variety of diseases and disorders. For example, peripheral nerve stimulation has been used to treat chronic pain syndrome, incontinence, and a number of other disorders, diseases, and symptoms.

Stimulators have been developed to provide therapy for a variety of treatments. A stimulator can include a control module (with a pulse generator), one or more leads, and an array of stimulator electrodes on each lead. The stimulator electrodes are in contact with or near the nerves, muscles, or other tissue to be stimulated. The pulse generator in the control module generates electrical pulses that are delivered by the electrodes to body tissue.

### BRIEF SUMMARY

One aspect is a system for stimulating a nerve. The system includes a cuff lead including a cuff configured for disposition around the nerve and a plurality of electrodes disposed on the cuff; and a control module configured to generate stimulation pulses.

The control module includes stimulation circuitry configured for generating stimulation pulses and delivering the stimulation pulses to the cuff lead for stimulation using the electrodes of the cuff lead, a memory having instructions stored thereon, and a processor coupled to the stimulation circuitry and the memory and configured to execute the instructions to perform actions. The actions include directing the stimulation circuitry to deliver a macroburst to the cuff lead for stimulation using the electrodes of the cuff lead, wherein the macroburst includes a plurality of microbursts, a microburst delay between two consecutive microbursts of the macroburst, and a macroburst delay following the plurality of microbursts, wherein each of the microbursts includes a plurality of pulses arranged in a temporal sequence, wherein each of the pulses has a pulse width of no more than 100 μs and a total amplitude of no more than 100 μA, and repeating, at least one time, the directing of the stimulation circuitry to deliver the macroburst.

In at least some aspects, at least one pair of the pulses is delivered simultaneously with the two pulses of each of the at least one pair having opposite polarity. In at least some aspects, at least one of the pulses is distributed between a plurality of the electrodes.

In at least some aspects, at least two of the microbursts of the macroburst are identical. In at least some aspects, all of the microbursts of the macroburst are identical. In at least some aspects, at least two of the microbursts include identical pulses but differ in temporal order of the pulses.

In at least some aspects, the macroburst has a duration of in a range of 1 to 5 minutes. In at least some aspects, a separation period between consecutive ones of the pulses of at least one of the microbursts is no more than 30 μs.

Another aspect is a system for programming a control module for stimulation of a nerve. The system includes a programming device that includes a display; a memory having instructions stored thereon; and a processor coupled to the display and the memory and configured to execute the instructions to perform actions. The actions include receiving user input defining a macroburst, wherein the macroburst includes a plurality of microbursts, a microburst delay between two consecutive microbursts of the macroburst, and a macroburst delay following the plurality of microbursts, wherein each of the microbursts includes a plurality of pulses arranged in a temporal sequence, wherein the user input includes a duration for each of the microburst delays, a duration of the macroburst delay, and, for each of the pulses, a pulse width, a selection of at least one electrode, and an amplitude for each electrode of the selected at least one electrode, wherein each of the pulse widths is no more than 100 μs and, for each of the pulses, a sum of the amplitudes is no more than 100 μA; and programming a control module, coupleable to the cuff lead, to stimulate a nerve according to the macroburst using the at least one selected electrode or the at least one indicated electrode.

In at least some aspects, at least one pair of the pulses is delivered simultaneously with the two pulses of each of the at least one pair having opposite polarity. In at least some aspects, at least one of the pulses is distributed between a plurality of the electrodes.

In at least some aspects, all of the microbursts of the macroburst are identical. In at least some aspects, at least two of the microbursts include identical pulses but differ in temporal order of the pulses. In at least some aspects, a separation period between consecutive ones of the pulses of at least one of the microbursts is no more than 30 μs.

A further aspect is a system for mapping a nerve. The system includes a cuff lead including a cuff configured for disposition around a nerve and a plurality of electrodes disposed on the cuff; a control module coupled or coupleable to the cuff lead and configured to generate stimulation pulses and deliver the stimulation pulses to the cuff lead for stimulation of the nerve; and a programming device. The programming device includes a display; a memory having instructions stored thereon; and a processor coupled to the display and the memory and configured to execute the instructions to perform actions. The actions include directing the control module to generate and deliver, through one or more of the electrodes of the cuff lead disposed around a nerve, stimulation pulses according to at least one nerve mapping paradigm, wherein the at least one nerve mapping paradigm includes a temporal sequence of pulses delivered through the electrodes of the cuff lead, wherein consecutive pulses in the temporal sequence are delivered through different electrodes; associating stimulation effects with the electrodes of the cuff lead based on the temporal sequence; and mapping the electrodes to one or more axons, fascicles, nerve fibers, tracts of the nerve, or peripheral organs based on the association of the stimulation effects with the electrodes of the cuff lead.

In at least some aspects, the at least one nerve mapping paradigm includes a horizontal/vertical vector mapping paradigm, wherein the temporal sequence includes a plurality of consecutive pulses applied to non-adjacent electrodes of a single set of electrodes, wherein the electrodes of the single set of electrodes are arranged for disposition circumferentially around the nerve while overlapping longitudinally.

In at least some aspects, the at least one nerve mapping paradigm includes an oblique vector mapping paradigm, wherein the temporal sequence includes at least one pair of consecutive pulses from electrodes of different sets of electrodes, wherein the electrodes of each set of electrodes are arranged for disposition circumferentially around the nerve while overlapping longitudinally and the electrodes of the different sets do not overlap longitudinally.

In at least some aspects, the at least one nerve mapping paradigm includes a cyclical mapping paradigm, wherein the temporal sequence includes pulses delivered by sequentially activating adjacent electrodes of a single set of the electrodes in a single circumferential direction.

In at least some aspects, the at least one nerve mapping paradigm includes an anode block paradigm, wherein the temporal sequence includes, at a plurality of times, a combination of a first pulse, having a first polarity, activating a first plurality of non-adjacent electrodes disposed circumferentially around the nerve and a second pulse, having a second polarity opposite the first polarity, activating one electrode of a second plurality of non-adjacent electrodes disposed circumferentially around the nerve with selection of the one electrode sequentially and temporally rotating between the electrodes of the second plurality.

In at least some aspects, for separation periods between consecutive pulses, durations of consecutive separation periods are different.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic view of one embodiment of an electrical stimulation system that includes a lead electrically coupled to a control module;

FIG. 2A is a schematic view of one embodiment of the control module of FIG. 1 configured and arranged to electrically couple to an elongated device;

FIG. 2B is a schematic view of one embodiment of a lead extension configured and arranged to electrically couple the elongated device of FIG. 2A to the control module of FIG. 1;

FIG. 3 is a schematic perspective view of one embodiment of a cuff with two sets of sixteen longitudinal electrodes each and two radial electrodes;

FIG. 4 is a schematic view of one embodiment of a cuff, flattened for illustrative purposes, with two sets of eight longitudinal electrodes each and two radial electrodes;

FIG. 5 illustrates a macroburst using graphs of amplitude versus time for eight electrodes;

FIG. 6 is a flowchart of one embodiment of a method for programming a control module for stimulation of a nerve;

FIG. 7 is a flowchart of one embodiment of a method for stimulating a nerve;

FIG. 8A illustrates mapping paradigms using graphs of amplitude versus time for sixteen electrodes;

FIG. 8B is a cross-sectional view of a nerve and eight electrodes of FIG. 8A illustrating the first four pulses of the mapping paradigms of FIG. 8A;

FIG. 8C is a cross-sectional view of a nerve and another eight electrodes of FIG. 8A illustrating the last five pulses of the mapping paradigms of FIG. 8A;

FIG. 9 illustrates a cyclical mapping paradigm using graphs of amplitude versus time for eight electrodes and a case of a control module;

FIG. 10 illustrates an anode block paradigm using graphs of amplitude versus time for eight electrodes and a case of a control module;

FIG. 11 is a flowchart of one embodiment of a method for mapping a nerve;

FIG. 12 illustrates one embodiment of an interface for programming a macroburst or a mapping paradigm; and

FIG. 13 is a schematic overview of one embodiment of components of an electrical stimulation arrangement according to an embodiment of the present invention.

### DETAILED DESCRIPTION

The present disclosure is directed to the area of peripheral nerve stimulation systems and methods of making and using the systems. The present disclosure is also directed to programming and stimulation using macrobursts, having multiple microbursts, or nerve mapping paradigms.

Suitable implantable electrical stimulation systems include, but are not limited to, a least one lead with one or more electrodes disposed along a distal end of the lead. Leads include, for example, percutaneous leads, paddle leads, and cuff leads. Examples of electrical stimulation systems with leads are found in, for example, U.S. Pat. Nos. 6,181,969; 6,516,227; 6,609,029; 6,609,032; 6,741,892; 7,203,548; 7,244,150; 7,450,997; 7,596,414; 7,610,103; 7,672,734; 7,761,165; 7,783,359; 7,792,590; 7,809,446; 7,949,395; 7,974,706; 6,175,710; 6,224,450; 6,271,094; 6,295,944; 6,364,278; 6,391,985; 7,596,414; 7,974,706; 8,423,157; 8,688,235; 8,831,742; 10,485,969; 10,493,269; 10,709,888; and 10,814,127; and; and U.S. Patent Applications Publication Nos. 2007/0150036; 2009/0187222; 2009/0276021; 2010/0076535; 2010/0268298; 2011/0004267; 2011/0078900; 2011/0130817; 2011/0130818; 2011/0238129; 2011/0313500; 2012/0016378; 2012/0046710; 2012/0071949; 2012/0165911; 2012/0197375; 2012/0203316; 2012/0203320; 2012/0203321; 2012/0316615; 2013/0105071; 2017/0333692; 2018/0154156; 2022/0226641; and 2022/0370793, all of which are incorporated by reference in their entireties.

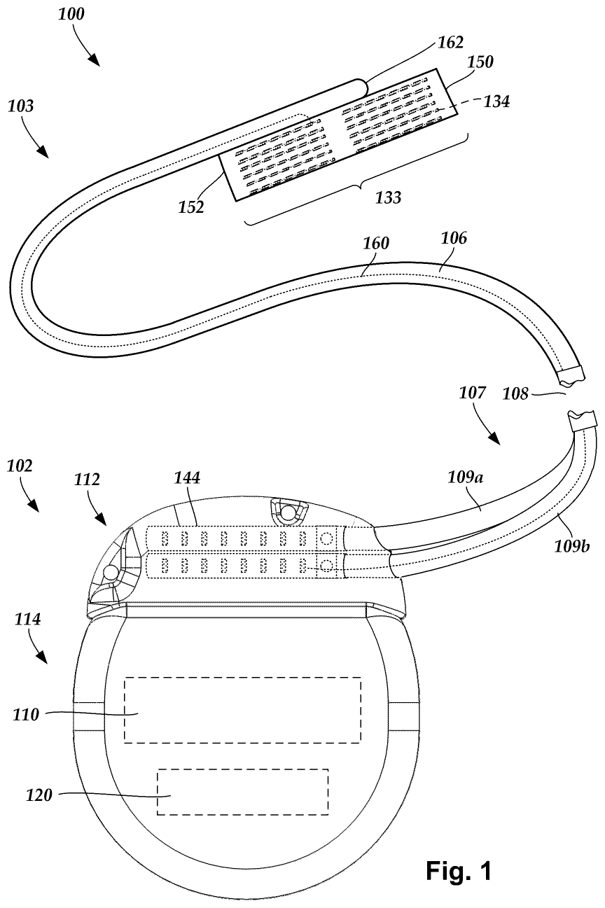

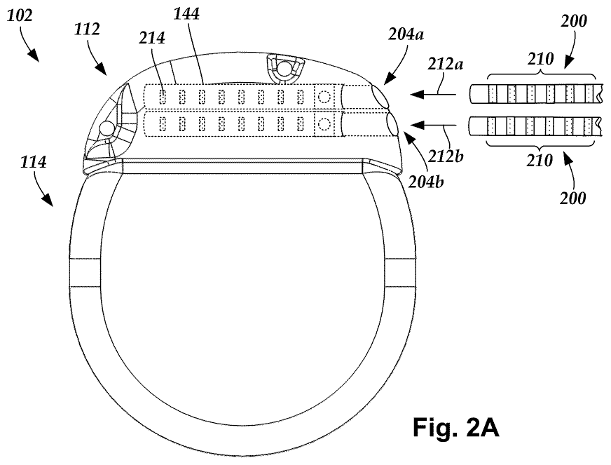

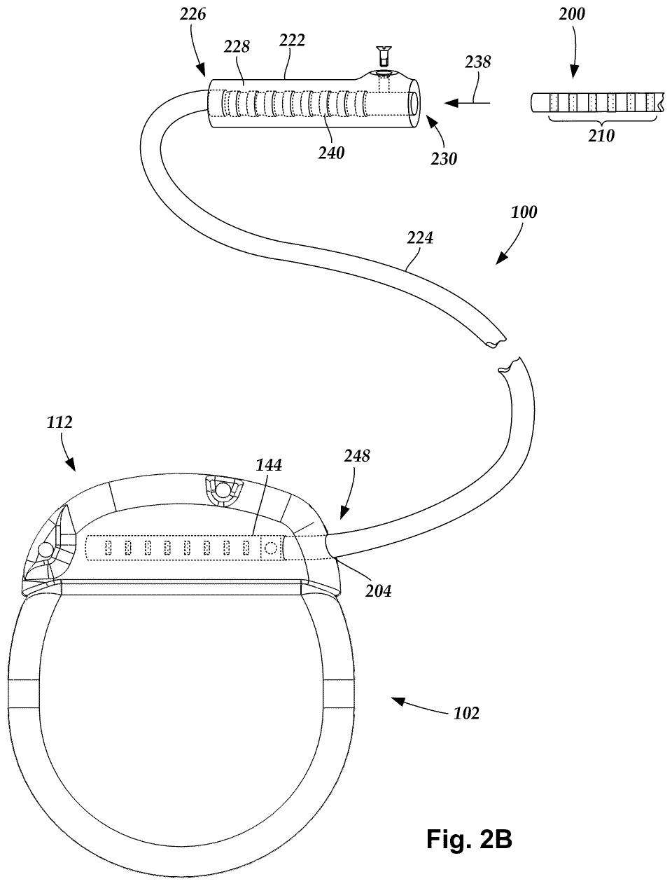

FIG. 1 illustrates schematically one embodiment of an electrical stimulation system 100. The electrical stimulation system includes a control module (e.g., a stimulator or pulse generator) 102 and a lead 103 coupleable to the control module 102. The lead 103 includes a mount 162 and a cuff 150 with a cuff body 152 and an array of electrodes 133, such as electrode 134. The lead 103 also includes one or more lead bodies 106, coupled to or containing the mount 162, and an array of terminals (e.g., 210 in FIG. 2A-2B) attached to the one or more lead bodies 106. In at least some embodiments, the lead is isodiametric along at least a portion of the longitudinal length of the lead body 106. FIG. 1 illustrates one lead 103 coupled to a control module 102. Other embodiments may include two, three, four, or more leads 103 coupled to the control module 102. In yet other embodiments, a lead 103 may be coupled to multiple control modules 102. For example, a lead with 64 electrodes may be coupled to two control modules 102 that are capable of handling 32 electrodes each.

The lead 103 can be coupled to the control module 102 in any suitable manner. In at least some embodiments, the lead 103 couples directly to the control module 102. In at least some other embodiments, the lead 103 couples to the control module 102 via one or more intermediate devices (200 in FIGS. 2A-2B). For example, in at least some embodiments one or more lead extensions 224 (see e.g., FIG. 2B) can be disposed between the lead 103 and the control module 102 to extend the distance between the lead 103 and the control module 102. Other intermediate devices may be used in addition to, or in lieu of, one or more lead extensions including, for example, a splitter, an adaptor, or the like or combinations thereof. It will be understood that, in the case where the electrical stimulation system 100 includes multiple elongated devices disposed between the lead 103 and the control module 102, the intermediate devices may be configured into any suitable arrangement.

In FIG. 1, the electrical stimulation system 100 is shown having a splitter 107 configured and arranged for facilitating coupling of the lead 103 to the control module 102. The splitter 107 includes a splitter connector 108 configured to couple to a proximal end of the lead 103, and one or more proximal tails 109a and 109b configured and arranged to couple to the control module 102 (or another splitter, a lead extension, an adaptor, or the like). The splitter 107 and splitter connector 108 may be part of the lead 103 or may be a separate component that attaches to the lead.

The control module 102 typically includes a connector housing 112 and a sealed electronics housing 114. Stimulation circuitry 110 and an optional power source 120 are disposed in the electronics housing 114. A control module connector 144 is disposed in the connector housing 112. The control module connector 144 is configured and arranged to make an electrical connection between the lead 103 and the stimulation circuitry 110 of the control module 102.

The electrical stimulation system or components of the electrical stimulation system, including the lead body 106 and the control module 102, are typically implanted into the body of a patient. The lead body 106 can be made of, for example, a non-conductive, biocompatible material such as, for example, silicone, polyurethane, polyetheretherketone (“PEEK”), epoxy, and the like or combinations thereof. The lead body 106 may be formed in the desired shape by any process including, for example, molding (including injection molding), casting, and the like. The non-conductive material typically extends from the distal end of the lead body 106 to the proximal end of the lead body 106.

Terminals (e.g., 210 in FIGS. 2A-2B) are typically disposed along the proximal end of the lead body 106 of the electrical stimulation system 100 (as well as any splitters, lead extensions, adaptors, or the like) for electrical connection to corresponding connector contacts (e.g., 214 and 240 in FIG. 2B). The connector contacts (e.g., 214 and 240 in FIG. 2B) are disposed in connectors (e.g., 144 in FIGS. 1-2B; and 222 in FIG. 2B) which, in turn, are disposed on, for example, the control module 102 (or a lead extension, a splitter, an adaptor, or the like). Electrically conductive wires 160, cables, or the like (only one of which is shown in FIG. 1) extend from the terminals to the electrodes 134. Typically, one or more electrodes 134 are electrically coupled to each terminal. In at least some embodiments, each terminal is only connected to one electrode 134.

The electrically conductive wires (“conductors”) 160 (only one of which is illustrated in FIG. 1 for clarity) may be embedded in the non-conductive material of the lead body 106 or can be disposed in one or more lumens (not shown) extending along the lead body 106. In some embodiments, there is an individual lumen for each conductor. In other embodiments, two or more conductors extend through a lumen. There may also be one or more lumens (not shown) that open at, or near, the proximal end of the lead body 106, for example, for inserting a stylet to facilitate placement of the lead body 106 within a body of a patient. Additionally, there may be one or more lumens (not shown) that open at, or near, the distal end of the lead body 106, for example, for infusion of drugs or medication into the site of implantation of the lead body 106. In at least one embodiment, the one or more lumens are flushed continually, or on a regular basis, with saline, epidural fluid, or the like. In at least some embodiments, the one or more lumens are permanently or removably sealable at the distal end.

FIG. 1 also illustrates a mount 162, part of the lead body 106, coupled to cuff 150. The conductors 160 (only one of which is illustrated in FIG. 1 for clarity) from within the lead body 106 are received in the mount 162, which in turn is attached to the cuff 150 such that each conductor passes through the mount 162 for a direct electrical connection with one of the electrodes 134 (e.g., one conductor is electrically connected with one electrode and so on). The mount 162 may be attached using a variety of means such as, but not limited to, molding or adhering the mount 162 to the cuff 150. In other embodiments, the conductors 160 from within the lead body 106 are electrically coupled to the electrodes 134 using jumper, intermediate or transition wires from the lead body 106 to the electrodes 134.

The mount 162 can be offset from the cuff 150, as illustrated in FIG. 1, or in-line with the cuff or in any other suitable arrangement. Non-limiting examples of cuff leads 103 can be found at U.S. Pat. Nos. 7,596,414; 7,974,706; 8,423,157; 10,485,969; 10,493,269; 10,709,888; and 10,814,127; U.S. Patent Application Publications Nos. 2017/0333692; 2018/0154156; 2022/0226641; and 2022/0370793 and U.S. Provisional Patent Applications Ser. Nos. 63/539,774 and 63/549,797, all of which are incorporated herein by reference in their entireties.

FIG. 2A is a schematic side view of one embodiment of a proximal end of one or more elongated devices 200 configured and arranged for coupling to one embodiment of the control module connector 144. The one or more elongated devices may include, for example, the lead body 106, one or more intermediate devices (e.g., the lead extension 224 of FIG. 2B, an adaptor, or the like or combinations thereof), or a combination thereof. FIG. 2A illustrates two elongated devices 200 coupled to the control module 102. These two elongated devices 200 can be two tails as illustrated in FIG. 1 or two different leads or any other combination of elongated devices.

The control module connector 144 defines at least one port into which a proximal end of the elongated device 200 can be inserted, as shown by directional arrow 212. In FIG. 2A (and in other figures), the connector housing 112 is shown having two ports 204a and 204b. The connector housing 112 can define any suitable number of ports including, for example, one, two, three, four, five, six, seven, eight, or more ports.

The control module connector 144 also includes a plurality of connector contacts, such as connector contact 214, disposed within each port 204a and 204b. When the elongated device 200 is inserted into the ports 204a and 204b, the connector contacts 214 can be aligned with a plurality of terminals 210 disposed along the proximal end(s) of the elongated device(s) 200 to electrically couple the control module 102 to the electrodes (134 of FIG. 1) disposed at a distal end of the lead 103. Examples of connectors in control modules are found in, for example, U.S. Pat. Nos. 7,244,150 and 8,224,450, which are incorporated by reference in their entireties.

FIG. 2B is a schematic side view of another embodiment of the electrical stimulation system 100. The electrical stimulation system 100 includes a lead extension 224 that is configured and arranged to couple one or more elongated devices 200 (e.g., the lead body 106, an adaptor, another lead extension, or the like or combinations thereof) to the control module 102. In FIG. 2B, the lead extension 224 is shown coupled to a single port 204 defined in the control module connector 144. Additionally, the lead extension 224 is shown configured and arranged to couple to a single elongated device 200. In alternate embodiments, the lead extension 224 is configured and arranged to couple to multiple ports 204 defined in the control module connector 144, or to receive multiple elongated devices 200, or both.

A lead extension connector 222 is disposed on the lead extension 224. In FIG. 2B, the lead extension connector 222 is shown disposed at a distal end 226 of the lead extension 224. The lead extension connector 222 includes a connector housing 228.

The connector housing 228 defines at least one port 230 into which terminals 210 of the elongated device 200 can be inserted, as shown by directional arrow 238. The connector housing 228 also includes a plurality of connector contacts, such as connector contact 240. When the elongated device 200 is inserted into the port 230, the connector contacts 240 disposed in the connector housing 228 can be aligned with the terminals 210 of the elongated device 200 to electrically couple the lead extension 224 to the electrodes (e.g., electrodes 134 of FIG. 1) disposed along the lead (e.g., lead 103 of FIG. 1).

In at least some embodiments, the proximal end of the lead extension 224 is similarly configured and arranged as a proximal end of the lead 103 (or other elongated device 200). The lead extension 224 may include a plurality of electrically conductive wires (not shown) that electrically couple the connector contacts 240 to a proximal end 248 of the lead extension 224 that is opposite to the distal end 226. In at least some embodiments, the conductive wires disposed in the lead extension 224 can be electrically coupled to a plurality of terminals (not shown) disposed along the proximal end 248 of the lead extension 224. In at least some embodiments, the proximal end 248 of the lead extension 224 is configured and arranged for insertion into a connector disposed in another lead extension (or another intermediate device). In other embodiments (and as shown in FIG. 2B), the proximal end 248 of the lead extension 224 is configured and arranged for insertion into the control module connector 144.

Any suitable arrangement of electrodes 134 on the cuff 150 can be used. Examples of cuff leads 103 and electrode arrangements for cuff leads can be found at U.S. Pat. Nos. 7,596,414; 7,974,706; 8,423,157; 10,485,969; 10,493,269; 10,709,888; and 10,814,127; U.S. Patent Application Publications Nos. 2017/0333692; 2018/0154156; 2022/0370793; and 2022/0395690; and U.S. Provisional Patent Applications Ser. Nos. 63/539,774 and 63/549,797, all of which are incorporated herein by reference in their entireties.

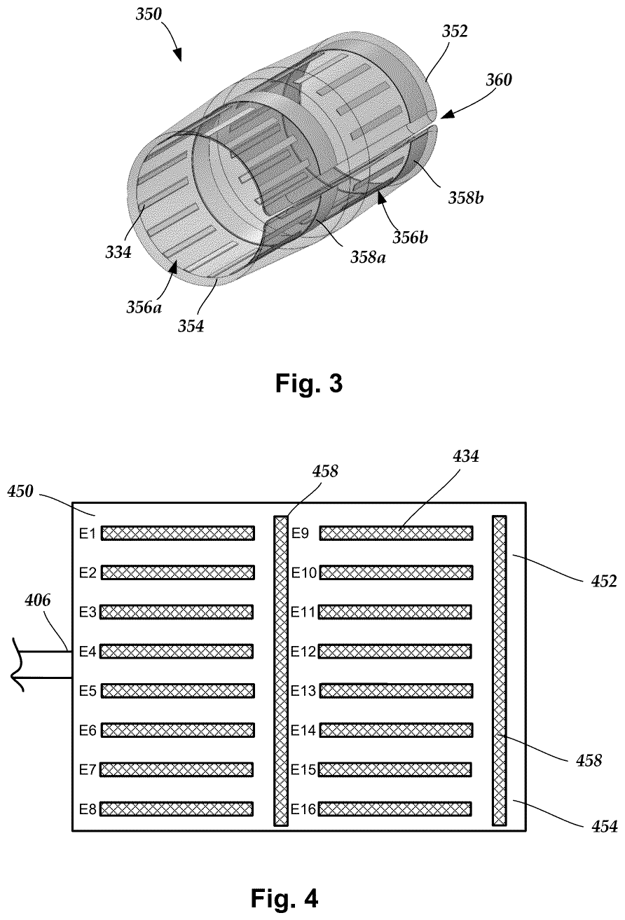

FIG. 3 illustrates one embodiment of a cuff 350 of a cuff lead 103 (FIG. 1). The cuff 350 includes a cuff body 352 with longitudinal electrodes 334 disposed on an interior surface 354 of the cuff body and arranged around the circumference of the cuff body in two sets 356a, 356b. In the illustrated embodiment, each set 356a, 356b includes sixteen longitudinal electrodes 334. Any other suitable number of electrodes can be used including, but not limited to, 16, 20, 25, 28, 32, 36, 40, 48, 50, 64, 80, 100, 120, 128, 150, 200, 250, 256, or more longitudinal electrodes. A cuff lead can have one, two, three, four, or more sets of longitudinal electrodes 334. The number of longitudinal electrodes 334 in a set can be the same for each set or can differ. In the illustrated embodiment, the longitudinal electrodes 334 of each set are aligned longitudinally with electrodes of the other set. In other embodiments, the longitudinal electrodes 334 of each set can be staggered or unaligned with the electrodes of another set.

In addition, the cuff 350 includes two radial electrodes 358a, 358b that wrap around at least 75%, 80%, 90%, or 95% of the circumference of the cuff body 352. The cuff 350 also defines a slit 360 that extends the longitudinal length of the cuff body 352 so that the nerve can be loaded into the interior 362 of the cuff body by opening the slit to fit the cuff body over the nerve. The slit 360 is opened or initially sized to allow the target nerve (not shown) to be slipped, inserted, fed, or otherwise received into the cuff 350 such that the cuff 350 wraps around the target nerve. In at least some embodiments, the slit 360 allows the cuff 350 to be easily moved over and around the target nerve or relative to the target nerve whether rotationally or transitionally.

The electrodes 134, 334, 358a, 358b can be formed using any conductive, biocompatible material. Examples of suitable materials include metals, alloys, conductive polymers, conductive carbon, and the like, as well as combinations thereof. In at least some embodiments, one or more of the electrodes 134, 334, 358a, 358b are formed from one or more of: platinum, platinum alloys such as platinum iridium, palladium alloys such as palladium rhodium, titanium, titanium alloys, nickel alloys, cobalt alloys, nickel/cobalt alloys, stainless steel, tantalum, conductive carbon, conductive plastics, epoxy or other adhesive filled with metallic powder, Nitinol™, or the like or any combination thereof. The electrodes 134, 334, 358a, 358b can be formed by any suitable process including, but not limited to, machining, molding (for example, powdered metal molding), photolithography, additive techniques, stamping, or the like or any combination thereof.

The cuff body 152, 352 can be formed of any suitable biocompatible and biostable non-conductive material including, but not limited to, polymer materials such as silicone, polyurethane, polyetheretherketone (“PEEK”), epoxy, or the like or any combination thereof. In at least some embodiments, the cuff body 152, 352 can have a circular, oval, or any other suitable cross-sectional shape and, at least in some embodiments, may be sufficiently flexible to alter the cross-sectional shape to accommodate the nerve. In at least some embodiments, the electrodes 134, 334, 358a, 358b can be molded with the cuff body 152, 352 or formed by techniques such as etching or ablation of conductive layers, films, or the like. In at least some embodiments, the cuff body 152, 352 has an inner diameter (which can correspond to the largest diameter of a non-circular cuff body) in a range of 0.5 to 5 mm or in a range of 1 to 3 mm. at least some embodiments, the cuff body 152, 352 has a length of at least 5, 10, or 20 mm.

In at least some embodiments, the cuff body 152, 352 can be formed using any suitable technique including, but not limited to, molding, casting, formed in a sheet and then shaped using adhesive as a binder, formed flat and shaped using heat, formed flat and attached to a cuff-shaped scaffold, pressed or extruded into the cuff shape, or the like or any combination thereof. In at least some embodiments, the electrodes 134, 334, 358a, 358b can be attached to the cuff body 152, 352 using any suitable technique including, but not limited to, attaching with adhesive, molding (for example, insert molding) into the cuff body, using heat to adhere the electrodes to the cuff body, heating and pressing the electrodes into the cuff body, depositing electrode material on the cuff body and using photolithography and etching, or the like or any combination thereof.

In at least some embodiments, once the cuff 350 has been placed in a desired position relative to the target nerve, the edges of the cuff body 352 defining the slit 360 can be sutured to capture the target nerve without undesirably compressing the target nerve. In at least some embodiments, suture holes (not shown) are optionally incorporated into the edges of the cuff 350 to allow for closing or partially closing the cuff 350 around the target nerve. Any other suitable arrangement, method, or technique can be used to secure the cuff 350 to the target nerve.

FIG. 4 illustrates another embodiment of a cuff 450 and distal end of a lead body 406 of a cuff lead 103 (FIG. 1). For illustrative purposes, the cuff 450 is illustrated unwrapped and laid flat. The cuff 450 includes a cuff body 452 with longitudinal electrodes 434 (labeled E1 to E16) disposed on an interior surface 454 of the cuff body and arranged around the circumference of the cuff body in two sets of eight electrodes each (electrodes E1 to E8 and electrodes E9 to E16, respectively). The cuff 450 also includes two radial electrodes 458.

A cuff of a cuff lead can be implanted around a peripheral nerve, such as the vagus nerve, to stimulate (e.g., by delivering electrical pulses) and either generate actions potentials or block transmission of action potentials to either the brain or to one or more peripheral organs or other nerve terminals. In at least some embodiments, such stimulation can be used to modulate the function of the brain, the peripheral organ(s), or both.

As an example, electrical stimulation or neuromodulation of the vagus nerve (or any other suitable nerve) can be used to alleviate various disease or disorder conditions of the brain, the peripheral organ(s), or other portions of the body. Conventional vagus nerve stimulation methods stimulate or modulate the nerve bundle indiscriminately. Because the vagus nerve innervates numerous organs, for example, the larynx, heart, lungs, spleen, and intestines, and carries information from all these organs to the brain, non-directional, non-target-specific stimulation or neuromodulation of the vagus nerve (or other nerve) can cause off-target effects or side effects.

In at least some embodiments, the methods and systems described herein can be used to recruit, stimulate, or modulate a nerve target such as, for example, one or more specific axons, fibers, fascicles, or tracts. In at least some embodiments, targeted stimulation can result in fewer (or no) off-target effects or side effects.

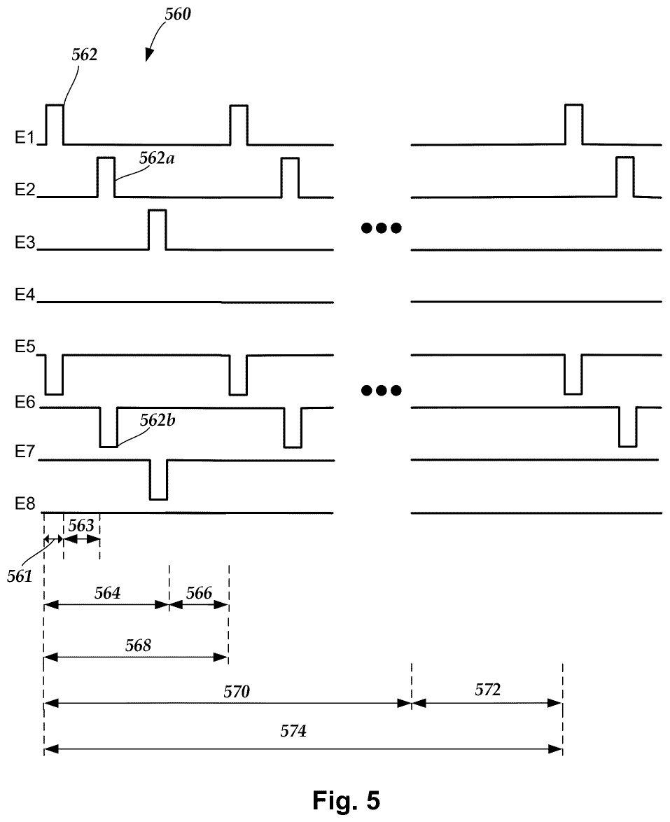

FIG. 5 illustrates a microbursting scheme that can be used, at least in some embodiments, for providing targeted therapeutic stimulation or modulation. In at least some embodiments, the therapeutic stimulation can be used to treat one or more disease or disorder conditions such as, for example, epilepsy (and its sub-types), treatment resistant depression (TRD), stroke, heart failure (HF), irritant bowel syndrome (IBS), immunological deficiencies, or the like or any combination thereof.

In at least some embodiments, the microbursting scheme can be used for identifying stimulation targets relative to the electrodes of the cuff or for mapping a nerve. In FIG. 5, a temporal pattern 560 of stimulation pulses 562 is presented for each of eight electrodes E1 to E8 (see, e.g., cuff 450 of FIG. 4). The temporal pattern 560 includes microbursts 564 of stimulation pulses 562.

The microbursting scheme includes a series of temporally-separated pulses 562 generated by the stimulation system and delivered through the electrodes of the cuff. A pulse can be applied to any number of electrodes including, but not limited to, one, two (see, FIG. 5), three, or more electrodes. In at least some embodiments, at a given time, a pulse of the same polarity can be applied to two or more electrodes. In at least some embodiments, at a given time, a pulse of one polarity (e.g., positive pulse 562a) can be applied to one, two, three, or more electrodes and a pulse of another polarity (e.g., negative pulse 562b) can be applied to one, two, three or more other electrodes.

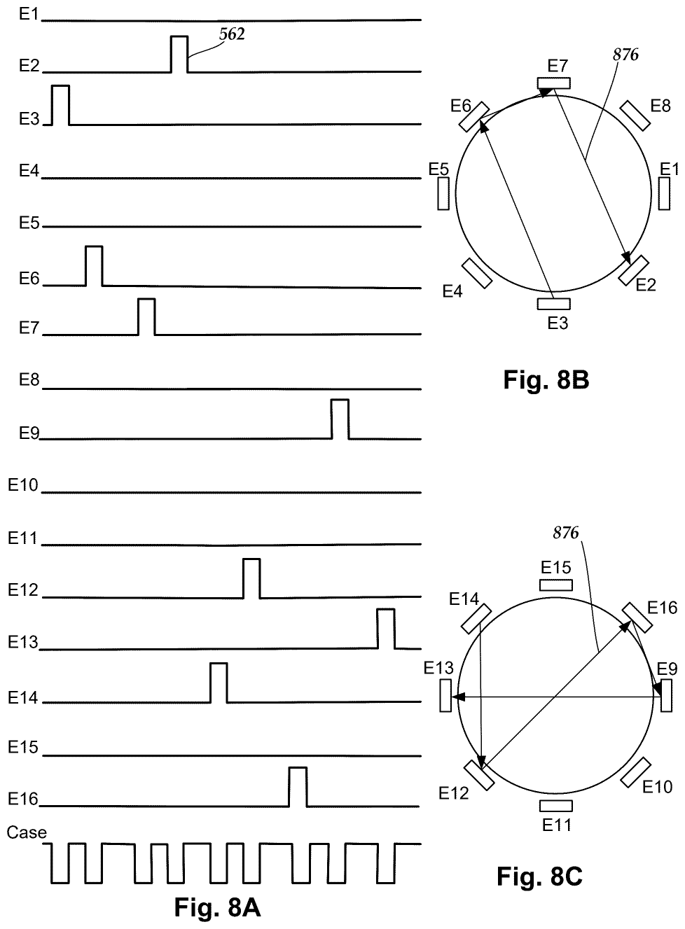

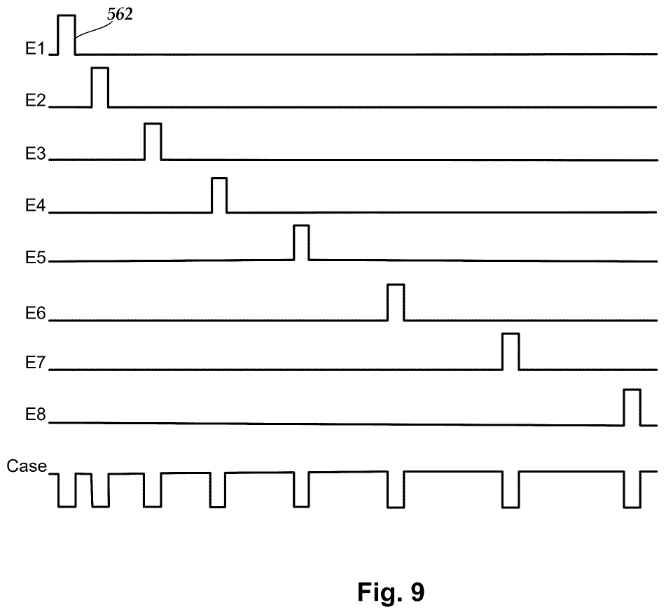

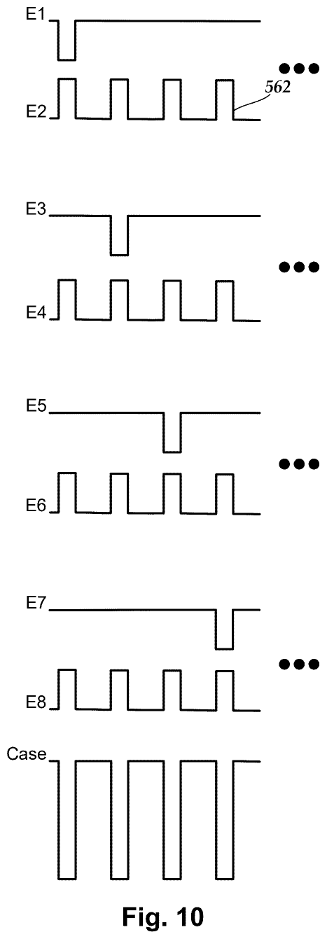

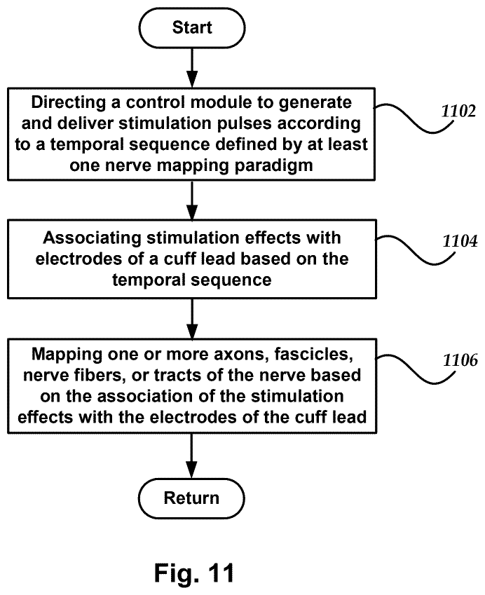

When a pulse is applied to multiple electrodes, the total amplitude of the pulse is distributed among the electrodes. The distribution can be described as “fractionalization”. In at least some embodiments, when a pulse is applied to multiple electrodes, the amplitude of the fractionalization of each electrode can be the same for some or all of the electrodes or can be different between two or more of the electrodes. In at least some embodiments, at any time, the stimulation provided by the cuff lead can be monopolar (see, for example, FIGS. 8A and 9), bipolar (see, for example, FIGS. 5 and 10), or multipolar (e.g., when three or more electrode are activated as a combination of alternating anodes/cathodes). In at least some embodiments, the stimulation can be anodic, cathodic, or a combination thereof. In at least some embodiments, the sealed electronics housing 114 (FIG. 1—“the case”) or other part of the control module 102 (FIG. 1) or cuff lead 103 (FIG. 1) that is relatively distant from the electrodes of the cuff 150 (FIG. 1) can act as an electrode (e.g., a return electrode), as illustrated in FIGS. 8A, 9, and 10. Although square pulses are illustrated in FIG. 5, it will be understood that any other suitable waveform can be used, such as for example, sinusoidal or triangular waveforms.

A microburst 564 includes a sequence of temporally-separated pulses 562, as illustrated in FIG. 1. A microburst 564 can have any suitable number of temporally-separated pulses 562 including, but not limited to, two, three, four, five, six, seven, eight, ten, twelve, or sixteen or more pulses. In FIG. 5, the microburst 564 includes the following sequence of three bipolar pairs of stimulation pulses: E1(positive)/E5(negative); E2(positive)/E6(negative); E3(positive)/E7(negative).

In at least some embodiments, when stimulation is targeted, a lower stimulation amplitude, as compared to the amplitude typically used for conventional nerve stimulation, can be used, particular when one or more electrodes proximal to the nerve target are selected. In at least some embodiments, each pulse 562 of a microburst 564 has an amplitude of at least 5, 10, or 20 μA. In at least some embodiments, each pulse 562 of a microburst 564 has an amplitude no greater than 1000, 500, 250, 100, 50, or 30 μA. In at least some embodiments, each pulse 562 of a microburst 564 has an amplitude in a range of 5 to 100 μA, 5 to 50 μA, 10 to 50 μA, or 10 to 30 μA. When a pulse is applied to multiple electrodes, this amplitude is distributed among the electrodes. In at least some embodiments, the pulses 562 of a microburst 564 have the same amplitude. In other embodiments, at least two of the pulses 562 of a microburst 564 have different amplitudes.

In at least some embodiments, pulses of different frequency can stimulate different types of axons, fibers, fascicles, or tracts. In at least some embodiments, changing the timing of the pulses or pulse patterns can facilitate specificity in recruiting, stimulating, or modulating specific axons, fibers, fascicles, or tracts. Targeting specific axons, fibers, fascicles, or tracts may also reduce or prevent overstimulation of an area of the nerve. In at least some embodiments, each pulse 562 has a pulse width 561 (i.e., duration) of at least 10, 20, or 30 μs. In at least some embodiments, each pulse 562 has a pulse width 561 (i.e., duration) of no more than 1 ms, 500 μs, 100 μs, 50 μs, or 30 μs. In at least some embodiments, each pulse 562 of a microburst 564 has a pulse width 561 (i.e., duration) in a range of 5 to 100 μs, 5 to 50 μs, or 10 to 30 μs. In at least some embodiments, the pulses 562 of a microburst 564 have the same pulse width. In other embodiments, at least two of the pulses 562 of a microburst 564 have different pulse widths.

In at least some embodiments, a separation period 563 between consecutive pulses 562 of a microburst 564 is at least 10, 20, 30, or 50 μs. In at least some embodiments, the separation period 563 between consecutive pulses 562 of a microburst 564 is no more than 500, 250, 100, or 50 μs. In at least some embodiments, the separation periods 563 between consecutive pulses 562 of a microburst 564 are the same. In at least some embodiments, the separation periods 563 between two pairs of consecutive pulses 562 of a microburst 564 are different. In at least some other embodiments, two or more pulses 562 of a microburst 564 can partially overlap in time. In at least some embodiments, each microburst 564 is followed by a microburst delay 566 (e.g., an intraburst delay). In at least some embodiments, the microburst delay is at least 10, 20, 30, 50, or 100 μs. In at least some embodiments, the microburst delay is no more than 2000, 1000, 500, 250, or 100 μs. In at least some embodiments, the microburst delay 566 is larger than the separation period 563 between consecutive pulses 562 of the microburst 564. In at least some embodiments, the microburst delay 566 is two, three, or four times larger than the separation period 563 between consecutive pulses of the microburst 564. In at least some embodiments, the microburst delay 566 for each of the microbursts 564 is the same. In other embodiments, the microburst delays 566 for at least two microbursts 564 are different.

The combination of the microburst 564 and microburst delay 566 defines the microburst duration 568. In at least some embodiments, the microburst duration 568 is at least 0.1, 0.5, 1, 5, 10, 20, 30, 50, 70, 90, 100, or 120 seconds. In at least some embodiments, the microburst duration 568 of each of the microbursts 564 is the same. In other embodiments, the microburst durations 568 of at least two microbursts 564 are different.

A macroburst 570 includes multiple microbursts 564, corresponding microburst delays 566, and a macroburst delay 572. The combination of the microburst durations 568 of the corresponding microbursts 564 and the macroburst delay 572 defines the macroburst duration 574. In at least some embodiments, a macroburst 570 can be repeated or a different microburst can be initiated. In at least some embodiments, each macroburst 570 has an identical sequence of microbursts 564 and stimulation pulses 562. In at least some embodiments, at least two consecutive macrobursts 570 have an identical sequence of microbursts 564 and stimulation pulses 562. In at least some embodiments, at least two consecutive macrobursts 570 have nonidentical sequences of microbursts 564 or stimulation pulses 562. In at least some embodiments, at least two consecutive macrobursts 570 have the same microbursts 564 or stimulation pulses 562 (i.e., identical in electrode selection and amplitude) in different temporal order.

In at least some embodiments, the macroburst duration 574, macroburst delay 572, or both are selected to reduce detrimental effects or side effects of continuous microburst stimulation. For example, in some instances, prolonged stimulation of a nerve may result in habituation of the nerve or result in the nerve no longer responding to the stimulation. As another example, in some instances, prolonged stimulation of the vagus nerve can result in esophageal constriction which can produce coughing or vomiting.

In at least some embodiments, the macroburst delay 572 is at least 10, 20, 30, 50, 60, 70, 90, 100, 120, 150, 180, 200, 210, or 240 seconds. In at least some embodiments, the macroburst delay 572 is no more than 1, 2, 3, 4, 5, 6, 7, 8, 9, 10, 12, 15, 20, or 30 minutes. In at least some embodiments, the macroburst delay 572 for each of the macrobursts 570 is the same. In other embodiments, the macroburst delays 572 for at least two macrobursts 570 are different.

In at least some embodiments, the macroburst duration 574 is at least 15, 20, 30, 50, 60, 70, 90, 100, 120, 150, 180, 200, 210, or 240 seconds. In at least some embodiments, the macroburst duration 574 is no more than 1, 2, 3, 4, 5, 6, 7, 8, 9, 10, 12, 15, 20, or 30 minutes. In at least some embodiments, the macroburst duration 574 is in a range of 15 seconds to 10 minutes or in a range of 30 seconds to 8 minutes or in a range of 1 to 5 minutes. In at least some embodiments, the macroburst duration 574 of each of the macrobursts 570 is the same. In other embodiments, the macroburst durations 574 of at least two macrobursts 570 are different.

In at least some embodiments, each microburst 564 of a macroburst 570 has an identical sequence of stimulation pulses 562. In at least some embodiments, at least two consecutive microbursts 564 of a macroburst 570 have an identical sequence (i.e., identical in electrode selection and amplitude) of stimulation pulses 562. In at least some embodiments, at least two consecutive microbursts 564 of a macroburst 570 have nonidentical sequences of stimulation pulses 562 (i.e., at least one corresponding pulse in each sequence differing in electrode selection or amplitude or any combination thereof). In at least some embodiments, at least two consecutive microbursts 564 of a macroburst 570 have the same pulses 562 (i.e., identical in electrode selection and amplitude) in different temporal order.

As one non-limiting example of a parameter selection for the macroburst illustrated in FIG. 5, in at least some embodiments, the amplitude is in a range of 5 to 100 μA, the pulse width is in a range of 10 to 100 μs, the microburst delay is in a range of 10 to 100 μs, the macroburst delay is at least 60 seconds, and the macroburst duration is in a range of 1 to 3 minutes.

In at least some embodiments, a reduction in stimulation amplitude for subsequent pulses 562 can be applied (for example, stepwise) at each microdelay (or set of microdelays) or each macrodelay (or set of macrodelays). In at least some embodiments, such a reduction can reduce the concentration of anodic (or cathodic) current over time. In at least some embodiments, this reduction of the concentration of anodic (or cathodic) current can create an anodic (or cathodic) block or an anodic (or cathodic) break excitation (e.g., an action potential generated by stoppage of hyperpolarizing current). Such a procedure may also facilitate understanding of the impact of stimulation on an organ or brain coupled to the stimulated nerve.

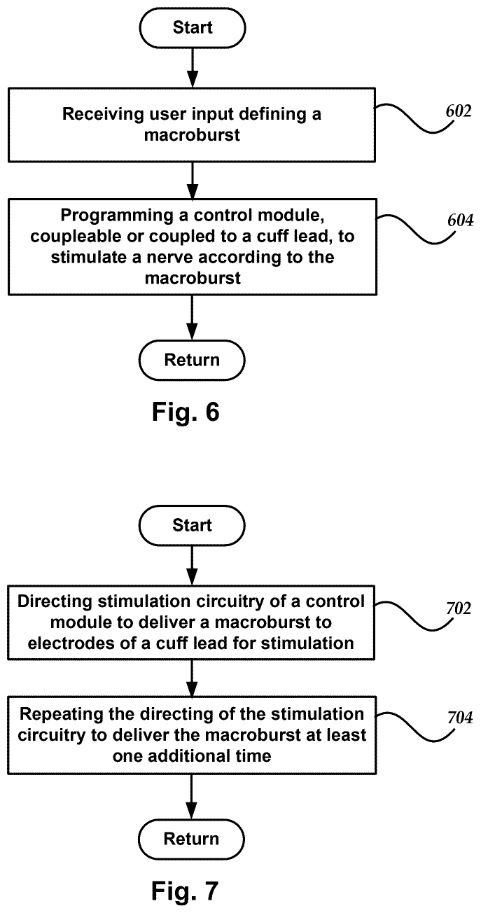

FIG. 6 is a flowchart of one embodiment of a method for programming a system for nerve stimulation. In step 602, user input is received for defining one or more macrobursts. The macroburst includes multiple microbursts and each microburst includes pulses arranged in a temporal sequence. The user input can include one or more of a duration for one or more microburst delays, a duration of a macroburst delay, a number of repetitions of the microburst in each macroburst, a number of macrobursts for the stimulation, or the like or any combination thereof. In addition, the user input can include, for each of the pulses, a pulse width, a selection of at least one electrode, and an amplitude for each electrode of the selected at least one electrode, or the like or any combination thereof. In step 604, a control module, coupleable or coupled to a cuff lead, is programmed to stimulate a nerve according to the macroburst.

FIG. 7 is a flowchart of one embodiment of a method for stimulating a nerve. In step 702, the stimulation circuitry of the control module is directed to generate and deliver a macroburst to electrodes of a cuff lead for stimulation of the nerve. In step 704, the direction to the stimulation circuit for delivery of the macroburst is repeated any number of times.

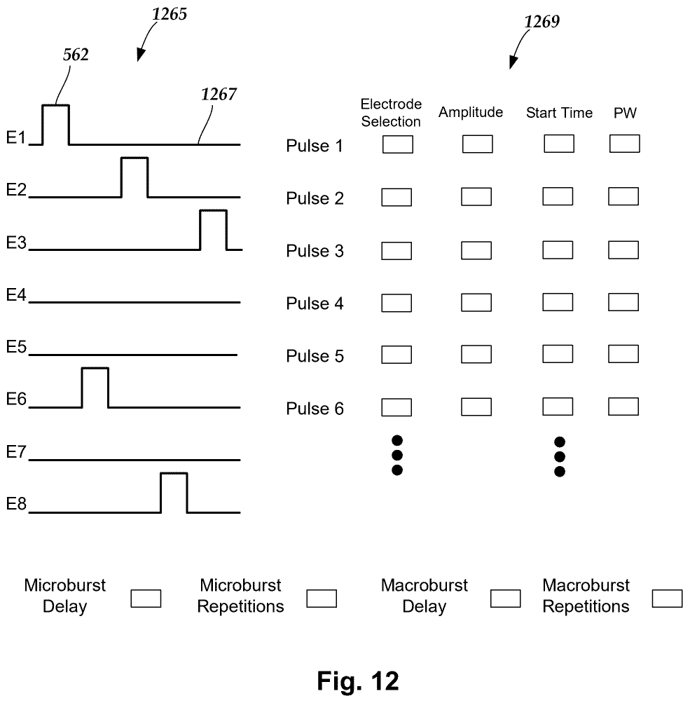

Any suitable method, interface, device, or system can be used for defining microbursts and macrobursts including any of the systems described in the references cited herein and in U.S. Provisional Patent Application Ser. No. 63/672,566, which is incorporated herein by reference in its entirety, as well as the interface of FIG. 12 described below. In at least some embodiments, an interface, device, or system allows a user to define pulses with one or more electrodes as cathodes or anodes and also define a time sequence of the pulses.

In at least some embodiments, adding or subtracting contacts around a selected contact can widen or narrow the stimulation field, respectively. In at least some embodiments, the stimulation field can be rotated clockwise or counterclockwise by adjusting the selection of electrodes and the fractionalization of the amplitude among the selected electrodes, as described in the references cited herein. In at least some embodiments, the stimulation field can be moved longitudinally along a cuff by selection of longitudinally-displaced electrodes, changes in fractionalization, or any combination thereof. Combining rotational and longitudinal movement can result in a spiral pattern of electrode activation along the cuff. In at least some embodiments, the stimulation field can be controlled in the longitudinal, rotational, or radial directions or any combination thereof.

In at least some embodiments, an interface, device, or system can include a set of predefined microburst or macroburst arrangements. In at least some embodiments, the interface, device, or system may individually associate some or all of these predefined arrangements with particular stimulation targets (e.g., body organs) and allow a user to select a stimulation target and receive an indication of one or more predefined microburst or macroburst arrangements for stimulation of that stimulation target. In at least some embodiments, an interface, device, or system can allow a user to select from multiple pre-defined spatial or temporal stimulation patterns. In at least some embodiments, the interface, device, or system includes a mechanism for determining or defining an orientation of the cuff and electrodes, after implantation, relative to features (e.g., axons, fascicles, nerve fibers, tracts, or the like) of the nerve.

In at least some embodiments, a method, device, or system, as described herein, can be used for mapping a nerve to facilitate identification of target axons, fascicles, nerve fibers, other nerve regions, or tracts for stimulation and which one or more electrodes can be used to stimulate the target. In at least some embodiments, the mapping can be performed for one or more quadrants, or any other suitable subdivision, of the nerve. In at least some embodiments, the mapping can provide selective fiber delineation in each quadrant or other subdivision. In at least some embodiments, the mapping can be used to identify, or associate with stimulation by one or more particular electrodes, one or more biomarker signals the fiber communicates to the brain and/or peripheral organs. In at least some embodiments, the mapping can be used for electrical modeling of one or more quadrants or other subdivisions of the nerve. In at least some embodiments, the mapping can be used for facilitating uni- or bi-directional stimulation of one or more target axons, fascicles, nerve fibers, other nerve regions, or tracts.

In at least some embodiments, the electrodes can be identified or grouped based on the axons, fascicles, nerve fibers, other nerve regions, or tracts that can be stimulated using the electrode or group of electrodes or peripheral organs or other regions of the body that can be affected by stimulation of the nerve. In at least some embodiments, the mapping can identify or estimate the position, proximity, or directionality (e.g., the direction to the nerve component of interest from the electrode(s)) of one or more of the axons, fascicles, nerve fibers, other nerve regions, or tracts relative to one or more of the electrodes. In at least some embodiments, the mapping can facilitate identifying one or more electrodes for stimulation of one or more target axons, fascicles, nerve fibers, other nerve regions, or tracts or for producing therapeutic effects in one or more peripheral organs of other regions of the body. In at least some embodiments, side effects arising from stimulation by one or more of the electrodes can be identified and inform the selection of one or more electrodes to stimulate one or more target axons, fascicles, nerve fibers, other nerve regions, or tracts or produce therapeutic effects in one or more peripheral organs of other regions of the body.

In at least some embodiments, a method, device, or system can include, or allow a user to define, one or more particular microburst or macroburst arrangements for mapping the nerve or portions of the nerve, for example, to identify one or more electrodes for stimulation of one or more target axons, fascicles, nerve fibers, other nerve regions, or tracts or for production of therapeutic effects in one or more peripheral organs of other regions of the body. In at least some embodiments, a method, device, or system can include incorporation of medical imaging (e.g., computed tomography (CT) imaging or magnetic resonance imaging (MRI)) or medical images to assist, facilitate, verify, or enhance the mapping.

The mapping includes stimulating the nerve with a temporal sequence of pulses by activating different electrodes. The stimulation can be, for example, monopolar (see, for example, FIGS. 8A and 9), bipolar (see, for example, FIG. 10), or multipolar or any combination thereof.

FIGS. 8A, 8B, and 8C illustrate examples of mapping paradigms using graphs of stimulation amplitude versus time for each electrode of a sixteen electrode cuff, such as the cuff 450 of FIG. 4. In the embodiment illustrated in FIG. 8A, the case (e.g., the sealed electronics housing 114 of FIG. 1) of the control module (e.g., the control module 102 of FIG. 1) acts as a return electrode.

FIGS. 8A, 8B, and 8C illustrate, for example, a horizontal/vertical vector mapping paradigm in which the stimulation transitions 876 between electrodes in the same set of electrodes (e.g., electrodes 1-8 or electrodes 9-16 of the cuff 450 of FIG. 4) correspond to transitions between non-adjacent electrodes of the set. In at least some embodiments, the stimulation transitions 876 of the horizontal/vertical vector mapping paradigm These stimulation transitions 876 can represent a horizontal or vertical transition or a vector having both horizontal and vertical components).

In at least some embodiments, a horizontal/vertical vector mapping paradigm includes one or more stimulation transitions 876 between two non-adjacent electrodes of the same set of electrodes. It will be recognized that any other suitable selection of two non-adjacent electrodes of the same set of electrodes can be used. Stimulation transitions 876 for the horizontal/vertical vector mapping paradigm illustrated in FIG. 8A are illustrated in FIGS. 8B and 8C. In FIG. 8B, the horizontal/vertical vector mapping paradigm includes stimulation transitions 876 from electrode E3 to electrode E6 and from electrode E7 to electrode E2, which correspond to the first four pulses 562 in FIG. 8A. In FIG. 8B, the horizontal/vertical vector mapping paradigm includes stimulation transitions 876 from electrode E14 to electrode E12; from electrode E12 to electrode E16; and from electrode E9 to electrode E13, which correspond to the last five pulses 562 in FIG. 8A. It will be recognized that any other suitable selection of two non-adjacent electrodes of the same set of electrodes can be used for the horizontal/vertical mapping paradigm.

FIG. 8A also illustrates an oblique vector mapping paradigm in which a stimulation transition 876 occurs between electrodes in different sets of electrodes, where those two electrodes are not longitudinally aligned with each other on the cuff. (For example, assuming the electrode arrangement of FIGS. 8B and 8C are aligned with each other, electrodes E1 and E9 are longitudinally aligned and electrodes E2 and E14 are not longitudinally aligned.) In FIG. 8A, the stimulation transition from E2 to E14 (the fourth and fifth pulses in FIG. 8A) and the stimulation transition from E13 to E3 (the last and first pulses in FIG. 8A), when the pattern in FIG. 8A is repeated, are stimulation transitions 876 of an oblique vector mapping paradigm.

In the horizontal/vertical and oblique mapping paradigms, the separation periods 563 (FIG. 5) for each pair of two consecutive pulses can be the same or different. For example, in the illustrated mapping paradigm of FIG. 8A, the separation periods 563 (FIG. 5) alternate between a first value (e.g., 30 μs) and a second value (e.g., 60 μs). In this illustrated embodiment, the second value is double the first value, but any other difference between the two values can be used. It will be recognized that any other selections of electrodes for oblique vector transitions can be used.

In at least some embodiments, the horizontal/vertical vector paradigm can be particularly useful for mapping, stimulating, or modulating myelinated fibers. In at least some embodiments, the oblique vector paradigm can be particularly useful for mapping, stimulating, or modulating unmyelinated fibers.

FIG. 9 illustrates a cyclical mapping paradigm. In the illustrated FIG. 9, the electrodes in a set of electrodes are activated sequentially around the circumference of the nerve. In the embodiment illustrated in FIG. 9, the sealed electronics housing (“the case”) of the control module acts as a return electrode. In the illustrated embodiment, the electrodes are activated starting with electrode E1 and ending with electrode E8. It will be understood that any of the electrodes can be the starting electrode and that the stimulation can proceed around the nerve clockwise or counter-clockwise. The cyclical mapping paradigm in FIG. 9 can be extended by activating electrodes E9 to E16 sequentially, starting with any of the electrodes in this set and proceeding clockwise or counter-clockwise.

In these mapping paradigms, the separation periods 563 (FIG. 5) for each pair of two consecutive pulses can be the same or different. In the illustrated mapping paradigm of FIG. 9, each consecutive separation period 563 (FIG. 5) is increased in time from the previous separation period. For example, the initial separation period 563 can be 30 μs and each consecutive separation period is increased by 30 μs from the immediately preceding separation period.

In at least some embodiments, shorter separation periods 563 can facilitate mapping, stimulation, or modulation of sensory fibers. In at least some embodiments, longer separation periods can facilitate mapping, stimulation, or modulation of motor fibers.

FIG. 10 illustrates an anode block paradigm in which, for each pulse, each of the even (or odd) electrodes are activated, as well as a rotating one of the odd (or even) electrodes is activated. In the illustrated embodiment, for each pulse, electrodes E2, E4, E6, and E8 are activated with a pulse of one polarity and one of electrodes E1, E3, E5, and E7 is activated, on a rotating basis, with a pulse of the opposing polarity. A different one of electrodes E1, E3, E5, and E7 is selected for consecutive pulses, where the selection of that electrode is rotated clockwise or counterclockwise around the nerve. In the illustrated embodiment, a portion of the opposite polarity amplitude is applied to the case (e.g., the sealed electronics housing 114 of FIG. 1) of the control module (e.g., the control module 102 of FIG. 1). In the illustrated embodiment, each of the electrodes is activated with the same amplitude and the amplitude applied to the sealed electronics housing (“the case”) of the control module is three times the electrode amplitude.

The illustrated embodiment of FIG. 10 is an anode block. It will be understood that a cathode block can also be provided by reversing the polarities of the pulses applied to the individual electrodes and case. An anode or cathode block can be used to block communication between the brain and one or more organs or other peripheral regions of the body.

Another example of an anode or cathode block (e.g., a “full ring block”) is the repeated application of a pulse of one polarity to each electrode (or a group of electrodes spaced around the circumference of the nerve) of one set of electrodes (for example, electrodes E1 to E8) and a pulse of the opposite polarity to each electrode (or a group of electrodes spaced around the circumference of the nerve) of the other set of electrodes (for example, electrodes E9 to E16). In some embodiments, the pulses of this anode or cathode block can be longer relative to the pulses illustrated in FIGS. 5, 8A, 9, and 10.

FIG. 11 is a flowchart of one embodiment of a method for mapping a nerve. In step 1102, a control module is directed to generate and deliver stimulation pulses according to a temporal sequence defined by at least one nerve mapping paradigm. In step 1104, stimulation effects are associated with electrodes of a cuff lead, through which the stimulation was delivered, according to the temporal sequence. For example, as stimulation occurs, any stimulation effect (for example, a therapeutic effect or side effect) is observed, measured, or identified and associated with the electrode(s) that was activated when the stimulation effect occurred. In step 1106, one or more axons, fascicles, nerve fibers, or tracts of the nerve or one or more peripheral organs based on the association of the simulation effects with the electrodes of the cuff lead.

FIG. 12 illustrates another embodiment of an interface 1265 for defining or illustrating the temporal sequence of the electrodes 334 of the cuff 350 for a macroburst, microburst, or mapping paradigm. This embodiment includes, for each of the electrodes 334, a graph 1267 of amplitude (or an on/off activation state) over time indicating electrode pulses 562. As illustrated in FIG. 12, these graphs 1267 can illustrate a time sequence of electrode activations. In FIG. 12, the electrodes are activated in the following order: electrode 1-electrode 6-electrode 2-electrode 8-electrode 3.

In at least some embodiments, the vertical axis of the graph indicates the amplitude of the current applied to the electrode. In at least some embodiments, the width of each electrode activation corresponds to the duration or pulse width of the activation. In at least some embodiments, two or more electrodes can be activated at any given time. In at least some embodiments, multiple electrodes can be activated at the same time and have different start times, stop times, or any combination thereof.

In at least some embodiments, a user can draw or modify the graphs 1267 and define the pulses 526 by the drawing. In at least some embodiments, the interface 1265 includes controls 1269 for entering one or more parameters for each pulse 562, such as, for example, electrode selection, start time of a pulse, stop time of a pulse, an amplitude at each electrode, a pulse width, or the like or any combination thereof. In at least some embodiments of an interface, for example, for defining a macroburst or microburst, other controls can be used for entering a microburst delay value, a number of microburst repetitions, a macroburst delay value, a number of macroburst repetitions, or the like or any combination thereof.

The interface of FIG. 12 is a non-limiting example of an interface for defining a macroburst and microbursts or a mapping paradigm. Any other suitable interface can be used for defining macrobursts and microbursts.

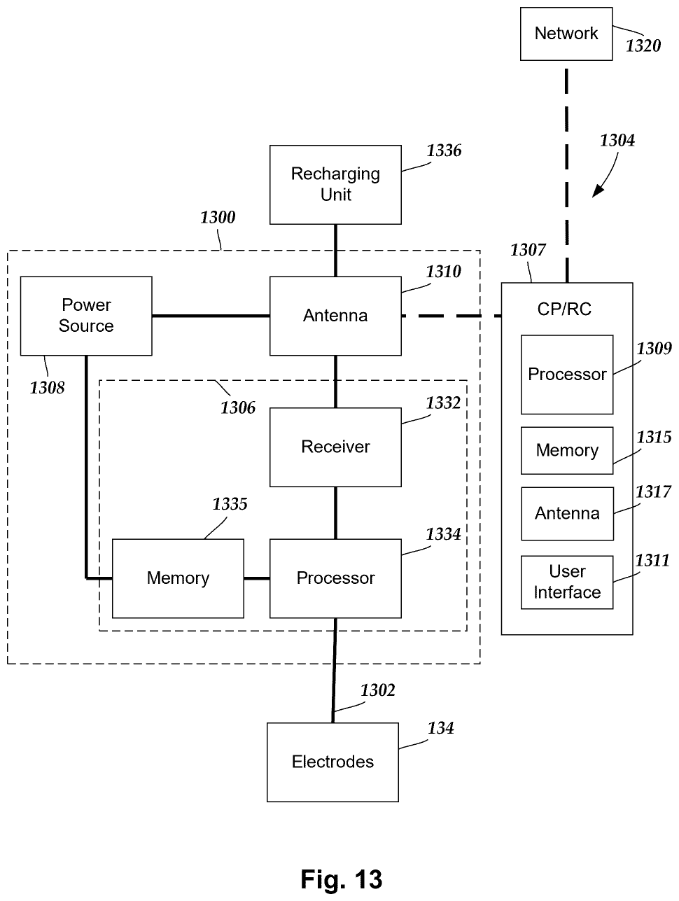

FIG. 13 is a schematic overview of one embodiment of components of an electrical stimulation system 1304 that includes a lead 1302 and a control module 1300 with stimulation circuitry 1306, a power source 1308, and an antenna 1310. The electrical stimulation system can be, for example, any of the electrical stimulation systems described above. It will be understood that the electrical stimulation system can include more, fewer, or different components and can have a variety of different configurations including those configurations disclosed in the stimulator references cited herein.

An external device, such as a clinician programmer (CP) or remote control (RC) 1307 (or any other suitable device or devices), can include a processor 1309, memory 1315, an antenna 1317, and a user interface 1311. The user interface 1311 can include, but is not limited to, a display screen on which a digital user interface can be displayed and any suitable user input device, such as a keyboard, touchscreen, mouse, track ball, or the like or any combination thereof.

Any power source 1308 can be used including, for example, a battery such as a primary battery or a rechargeable battery. Examples of other power sources include super capacitors, nuclear or atomic batteries, mechanical resonators, infrared collectors, thermally-powered energy sources, flexural powered energy sources, bioenergy power sources, fuel cells, bioelectric cells, osmotic pressure pumps, and the like including the power sources described in U.S. Pat. No. 7,437,1123, incorporated herein by reference in its entirety.

If the power source 1308 is a rechargeable battery or chargeable capacitor, the power source may be recharged/charged using the antenna 1310, if desired. Power can be provided for recharging/charging by inductively coupling the power source 1308 through the antenna 1310 to a recharging unit 1336 external to the user. Examples of such arrangements can be found in the references identified above.

In at least some embodiments, electrical current is emitted by the electrodes (such as electrodes 134 in FIG. 1) on the lead 1302 to stimulate nerve fibers, muscle fibers, or other body tissues near the electrical stimulation system. The stimulation circuitry 1306 can include, among other components, a processor 1334, a memory 1335, and a receiver 1332. The processor 1334 is generally included to control the timing and electrical characteristics of the electrical stimulation system. For example, the processor 1334 can, if desired, control one or more of the timing, frequency, strength, duration, and waveform of the pulses. In addition, the processor 1334 can select which electrodes can be used to provide stimulation, if desired. In some embodiments, the processor 1334 selects which electrode(s) are cathodes and which electrode(s) are anodes.

Any processor 1334 can be used and can be as simple as an electronic device that, for example, produces pulses at a regular interval or the processor can be capable of receiving and interpreting instructions from the CP/RC 1307 that, for example, allows modification of pulse characteristics. In the illustrated embodiment, the processor 1334 is coupled to a receiver 1332 which, in turn, is coupled to the antenna 1310. This allows the processor 1334 to receive instructions from an external source to, for example, direct the pulse characteristics and the selection of electrodes, if desired. Any suitable processor 1309 can be used for the CP/RC 1307.

Any suitable memory 1335, 1315 can be used including computer-readable storage media may include, but is not limited to, volatile, nonvolatile, non-transitory, removable, and non-removable media implemented in any method or technology for storage of information, such as computer readable instructions, data structures, program modules, or other data. Examples of computer-readable storage media include, but are not limited to, RAM, ROM, EEPROM, flash memory, or other memory technology, CD-ROM, digital versatile disks (“DVD”) or other optical storage, magnetic cassettes, magnetic tape, magnetic disk storage or other magnetic storage devices, or any other medium which can be used to store the desired information and which can be accessed by a processor.

In at least some embodiments, the antenna 1310 is capable of receiving signals (e.g., RF signals) from an antenna 1317 of a CP/RC 1307, which is programmed or otherwise operated by a user. The CP/RC 1307 can be a device that is worn on the skin of the user or can be carried by the user and can have a form similar to a pager, cellular phone, or remote control, if desired. As another alternative, the CP/RC 1307 may not be worn or carried by the user but may only be available at a home station or at a clinician's office.

The signals sent to the processor 1334 via the antenna 1310 and the receiver 1332 can be used to modify or otherwise direct the operation of the control module 1300. For example, the signals may be used to modify the pulses of the electrical stimulation system such as modifying one or more of pulse duration, pulse frequency, pulse waveform, and pulse strength. The signals may also direct the control module 1300 to cease operation, to start operation, t to start signal acquisition, to stop signal acquisition, to start charging the battery, or to stop charging the battery.

Optionally, the control module 1300 may include a transmitter (not shown) coupled to the processor 1334 and the antenna 1310 for transmitting signals back to the CP/RC 1307 or another unit capable of receiving the signals. For example, the control module 1300 may transmit signals indicating whether the control module 1300 is operating properly or not or indicating when the battery needs to be charged or the level of charge remaining in the battery. The processor 1334 may also be capable of transmitting information about the pulse characteristics so that a user or clinician can determine or verify the characteristics.

It will be understood that each block of the flowchart illustration, and combinations of blocks in the flowchart illustration and methods disclosed herein, can be implemented by computer program instructions. These program instructions may be provided to a processor to produce a machine or engine, such that the instructions, which execute on the processor, create means for implementing the actions specified in the flowchart block or blocks or engine disclosed herein. The computer program instructions may be executed by a processor to cause a series of operational steps to be performed by the processor to produce a computer implemented process. The computer program instructions may also cause at least some of the operational steps to be performed in parallel. Moreover, some of the steps may also be performed across more than one processor, such as might arise in a multi-processor computing device. In addition, one or more processes may also be performed concurrently with other processes, or even in a different sequence than illustrated without departing from the scope or spirit of the invention.

The computer program instructions can be stored on any suitable computer-readable medium including, but not limited to, RAM, ROM, EEPROM, flash memory or other memory technology, CD-ROM, digital versatile disks (“DVD”) or other optical storage, magnetic cassettes, magnetic tape, magnetic disk storage or other magnetic storage devices, or any other medium which can be used to store the desired information and which can be accessed by a computing device. The computer program instructions can be stored locally or nonlocally (for example, in the Cloud).

The above specification provides a description of the structure, manufacture, and use of the invention. Since many embodiments of the invention can be made without departing from the spirit and scope of the invention, the invention also resides in the claims hereinafter appended.

What is claimed as new and desired to be protected is:

1. A system for stimulating a nerve, the system comprising:
a cuff lead comprising a cuff configured for disposition around the nerve and a plurality of electrodes disposed on the cuff; and
a control module configured to generate stimulation pulses, the control module comprising
stimulation circuitry configured for generating stimulation pulses and delivering the stimulation pulses to the cuff lead for stimulation using the electrodes of the cuff lead,
a memory having instructions stored thereon, and
a processor coupled to the stimulation circuitry and the memory and configured to execute the instructions to perform actions, the actions comprising
directing the stimulation circuitry to deliver a macroburst to the cuff lead for stimulation using the electrodes of the cuff lead, wherein the macroburst comprises a plurality of microbursts, a microburst delay between two consecutive microbursts of the macroburst, and a macroburst delay following the plurality of microbursts, wherein each of the microbursts comprises a plurality of pulses arranged in a temporal sequence, wherein each of the pulses has a pulse width of no more than 100 μs and a total amplitude of no more than 100 μA, and
repeating, at least one time, the directing of the stimulation circuitry to deliver the macroburst.

2. The system of claim 1, wherein at least one pair of the pulses is delivered simultaneously with the two pulses of each of the at least one pair having opposite polarity.

3. The system of claim 1, wherein at least one of the pulses is distributed between a plurality of the electrodes.

4. The system of claim 1, wherein at least two of the microbursts of the macroburst are identical.

5. The system of claim 1, wherein all of the microbursts of the macroburst are identical.

6. The system of claim 1, wherein at least two of the microbursts comprise identical pulses but differ in temporal order of the pulses.

7. The system of claim 1, wherein the macroburst has a duration of in a range of 1 to 5 minutes.

8. The system of claim 1, wherein a separation period between consecutive ones of the pulses of at least one of the microbursts is no more than 30 μs.

9. A system for programming a control module for stimulation of a nerve, the system comprising:
a programming device comprising
a display;
a memory having instructions stored thereon; and
a processor coupled to the display and the memory and configured to execute the instructions to perform actions, the actions comprising:
receiving user input defining a macroburst, wherein the macroburst comprises a plurality of microbursts, a microburst delay between two consecutive microbursts of the macroburst, and a macroburst delay following the plurality of microbursts, wherein each of the microbursts comprises a plurality of pulses arranged in a temporal sequence, wherein the user input comprises a duration for each of the microburst delays, a duration of the macroburst delay, and, for each of the pulses, a pulse width, a selection of at least one electrode, and an amplitude for each electrode of the selected at least one electrode, wherein each of the pulse widths is no more than 100 μs and, for each of the pulses, a sum of the amplitudes is no more than 100 μA; and
programming a control module, coupleable to the cuff lead, to stimulate a nerve according to the macroburst using the at least one selected electrode or the at least one indicated electrode.

10. The system of claim 9, wherein at least one pair of the pulses is delivered simultaneously with the two pulses of each of the at least one pair having opposite polarity.

11. The system of claim 9, wherein at least one of the pulses is distributed between a plurality of the electrodes.

12. The system of claim 9, wherein all of the microbursts of the macroburst are identical.

13. The system of claim 9, wherein at least two of the microbursts comprise identical pulses but differ in temporal order of the pulses.

14. The system of claim 9, wherein a separation period between consecutive ones of the pulses of at least one of the microbursts is no more than 30 μs.

15. A system for mapping a nerve, the system comprising:
a cuff lead comprising a cuff configured for disposition around a nerve and a plurality of electrodes disposed on the cuff;
a control module coupled or coupleable to the cuff lead and configured to generate stimulation pulses and deliver the stimulation pulses to the cuff lead for stimulation of the nerve; and
a programming device comprising
a display;
a memory having instructions stored thereon; and
a processor coupled to the display and the memory and configured to execute the instructions to perform actions, the actions comprising:
directing the control module to generate and deliver, through one or more of the electrodes of the cuff lead disposed around a nerve, stimulation pulses according to at least one nerve mapping paradigm, wherein the at least one nerve mapping paradigm comprises a temporal sequence of pulses delivered through the electrodes of the cuff lead, wherein consecutive pulses in the temporal sequence are delivered through different electrodes;
associating stimulation effects with the electrodes of the cuff lead based on the temporal sequence; and
mapping the electrodes to one or more axons, fascicles, nerve fibers, tracts of the nerve, or peripheral organs based on the association of the stimulation effects with the electrodes of the cuff lead.

16. The system of claim 15, wherein the at least one nerve mapping paradigm comprises a horizontal/vertical vector mapping paradigm, wherein the temporal sequence comprises a plurality of consecutive pulses applied to non-adjacent electrodes of a single set of electrodes, wherein the electrodes of the single set of electrodes are arranged for disposition circumferentially around the nerve while overlapping longitudinally.

17. The system of claim 15, wherein the at least one nerve mapping paradigm comprises an oblique vector mapping paradigm, wherein the temporal sequence comprises at least one pair of consecutive pulses from electrodes of different sets of electrodes, wherein the electrodes of each set of electrodes are arranged for disposition circumferentially around the nerve while overlapping longitudinally and the electrodes of the different sets do not overlap longitudinally.

18. The system of claim 15, wherein the at least one nerve mapping paradigm comprises a cyclical mapping paradigm, wherein the temporal sequence comprises pulses delivered by sequentially activating adjacent electrodes of a single set of the electrodes in a single circumferential direction.

19. The system of claim 15, wherein the at least one nerve mapping paradigm comprises an anode block paradigm, wherein the temporal sequence comprises, at a plurality of times, a combination of a first pulse, having a first polarity, activating a first plurality of non-adjacent electrodes disposed circumferentially around the nerve and a second pulse, having a second polarity opposite the first polarity, activating one electrode of a second plurality of non-adjacent electrodes disposed circumferentially around the nerve with selection of the one electrode sequentially and temporally rotating between the electrodes of the second plurality.

20. The system of claim 15, wherein, for separation periods between consecutive pulses, durations of consecutive separation periods are different.
